(19) United States
(12) Patent Application Publication
(10) Pub. No.: US 2012/0276225 A1
(43) Pub. Date: Nov. 1, 2012

(54) PHYTOESTROGENIC NUTRACEUTICAL COMPOSITION FROM PALM LEAF EXTRACT

(75) Inventors: Suhaila Mohamed, Serdang (MY); Farideh Namvar, Serdang (MY)

(73) Assignee: UNIVERSITI PUTRA MALAYSIA, UPM Serdang, Selangor Darul Ehsan (MY)

(21) Appl. No.: 13/260,184

(22) PCT Filed: Mar. 24, 2009
(86) PCT No.: PCT/MY2009/000040

§ 371 Date: Dec. 8, 2011

(51) Int. Cl. A61K 36/889; A61P 9/00; A61P 3/04; A61P 25/00; A61P 13/00; A61P 15/00; A61P 19/10

(52) U.S. Cl. 424/727

(57) ABSTRACT

A comestible composition with phytoestrogenic property against oxidative stress, retarding hormone related cancer, affecting fertility and effective to hinder or prevent ailments or conditions resulting from, or exacerbated by, a decrease in endogenous estrogen including: reproductive organ ailments, cardiovascular disease, osteoporosis, loss of cognitive function, urinary incontinence, body fat increase, post menopausal syndromes and vasomotor symptoms, and contains extract from palm leaf.

### FIELD OF INVENTION

The present invention relates to a nutraceutical with significant phytoestrogenic property. The disclosed comestible composition can lengthen the estrus phase of the estrous cycle and have phytoestrogenic properties that protect against oxidative stress, retarding hormone related cancer, affecting fertility and effective to hinder or prevent ailments or conditions resulting from, or exacerbated by, a decrease in endogenous estrogen including: reproductive organ ailments, cardiovascular disease, osteoporosis, loss of cognitive function, urinary incontinence, body fat increase, post menopausal syndromes and vasomotor symptoms, and contains extract from palm leaf.

### BACKGROUND OF INVENTION

Phytoestrogens, [phyto (plant), estrus (period of fertility for female mammals)+gen (to generate)] are non steroidal plant compounds that are structurally similar to estradiol (17β-estradiol), and have the ability to cause estrogenic or/and antiestrogenic effects. Phytoestrogens are protective against diverse health disorders such as prostate, breast, bowel, and other cancers, cardiovascular disease, brain function disorders, menopausal symptoms and osteoporosis. Phytoestrogens interact with Estrogen Receptors (ER), and may modulate the endogenous estrogens concentration by binding or inactivating some enzymes, thus may affect the bioavailability of sex hormones through binding or stimulating the sex hormone binding globuline (SHBG) synthesis. Seeds contained the highest phytoestrogen contents. In human beings, phytoestrogens are readily absorbed, circulated in plasma and excreted in the urine. Theoretically, men exposed to high levels of phytoestrogens may have altered hypothalamic-pituitary-gonadal axis, however, such hormonal effects are small. Dietary phytoestrogen have no effect in sperm count or mobility or on testicular or ejaculate volume. Phytoestrogens may prevent prostate cancer. Epidemiological evidence showed phytoestrogens are protective against breast cancer. In cell line studies, low concentration of genistein supported tumor cell growth, but higher concentrations produced inhibitory effects. Phytoestrogens have different effects on fertility with different animals. Metabolic influence is different between ruminants, birds and monogastric mammals.

Metabolites from some plants have been proven to possess phytoestrogenic enhancing properties. A few phytoestrogenic enhancing products containing plant metabolites as the active ingredients have been developed. For example, U.S. Pat. No. 7,045,155 provides compositions enriched with natural phyto-oestrogens or analogues thereof selected from Genistein, Daidzein, Formononetin and Biochanin A. These may be used as food additives, tablets or capsules for promoting health in cases of cancer, pre-menstrual syndrome, menopause or hypercholesterolaemia.

U.S. Pat. No. 6,861,079 discloses a fertility kit and method for enhancing the natural fertility process. The kit includes a vaginal douche that is used prior to intercourse to enhance the sperm transportation and sustaining properties of the cervical mucous. The douche contains a balanced electrolyte solution, polysaccharides, and pH buffers. The kit also includes nutraceuticals specifically formulated for both the male and female which include amino acids, minerals, vitamins, herbs, phytoestrogens, and antioxidants along with a specified dosing regimen. A basal body temperature thermometer and chart is provided with instructions to confirm when and if ovulation will/did occur. Commercially available urinary chemical reagent strips are provided with instructions so as to predict/confirm if and when ovulation will occur. A lubricating medium will also be provided and utilized at the time of intercourse which is nonspermicidal and which contains polysaccharides which influence natural sperm motility. A detailed instruction book regarding the method and practice is provided along with dietary and lifestyle recommendations which have been shown to affect natural fertility.

KR0039720050214, provides a therapeutic agent for osteoporosis, comprising Ginkgo biloba leaf extract as an active ingredient. The Ginkgo biloba leaf extract contains a large amount of phytoestrogen such as quercetin and kaempferol and thus can be usefully utilized as a therapeutic agent for treatment of osteoporosis without having adverse effects of causing breast cancer due to capability to function as a selective estrogen receptor modulator.

US 20080167278 claims a composition comprising a progestin, a phytoestrogen compound, and an estrogen, in which the phytoestrogen is preferably an isoflavone compound selected from the group consisting of daidzein, genistein, and glycitein and their conjugated forms. The progestin is preferably selected from the group consisting of norgestimate, levonorgestrel, norgestrel, norethindrone, desogestrel, gestodene, dienogest, drospirenone, and medroxy-progesterone acetate. The estrogen is preferably from the group consisting of ethinyl estradiol, 17.alpha.-ethinylestradiol, and 17 beta-estradiol and conjugated estrogens.

EP1453827 provides methods for preparing an estrogenic preparation and isolating estrogenic compounds from a plant, such as an Epimedium plant, are provided. Also provided are estrogenic compounds from Epimedium plant that have been isolated and characterized and methods for their use in modulating estrogen receptors and in treating conditions mediated by estrogen receptors, such as menopause and estrogen-dependent cancers. Also provided are preparations from Epimedium that are enriched for estrogenic compounds, and methods for their use in modulating estrogen receptors and preventing and treating conditions that are mediated by estrogen receptors.

U.S. Pat. No. 6,326,366 discloses a hormone replacement therapy and a composition useful for women having reduced levels of endogenous estrogen. A mammalian estrogen and an isoflavone which is incapable of being metabolized to equol are co-administered to a woman having a reduced level of endogenous estrogen. The hormone replacement therapy is effective to inhibit or prevent diseases or conditions resulting from, or exacerbated by, a reduction in endogenous estrogen including: coronary heart disease, cardiovascular disease, osteoporosis, loss of cognitive function, urinary incontinence, weight gain, fat mass gain, and vasomotor symptoms. A composition for use in the hormone replacement therapy of the present invention contains a mammalian estrogen and at least one isoflavone, where the isoflavone is incapable of being metabolized to equol by a human and where the composition contains less than 10% by weight of isoflavones and phytoestrogens capable of being metabolized to equol by a human.

### SUMMARY OF INVENTION

The present invention aims to provide a nutraceutical composition which is effective in lengthening the estrus period of the estrous cycle and have phytoestrogenic properties that protect against oxidative stress, retarding hormone related cancer and effective to hinder or prevent ailments or conditions resulting from, or exacerbated by, a decrease in endogenous estrogen including: reproductive organ ailments, cardiovascular disease, osteoporosis, loss of cognitive function, urinary incontinence, body fat increase, and vasomotor symptoms which contains extract from palm leaf.

At least one of the preceding objects is met, in whole or in part, by the present invention, in which one of the embodiment of the present invention a composition with phytoestrogenic property comprising aqueous or alcoholic extract from palm leaf.

The present invention consists of several novel features and a combination of parts hereinafter fully described and illustrated in the accompanying description and drawings, it being understood that various changes in the details may be made without departing from the scope of the invention or sacrificing any of the advantages of the present invention.

### BRIEF DESCRIPTION OF THE DRAWINGS

The present invention will be fully understood from the detailed description given herein below and the accompanying drawings which are given by way of illustration only, and thus are not limitative of the present invention, wherein:

FIG. 1 is a graph showing the effect of PALM LEAVES EXTRACT on estrous cycle and estrus phase.

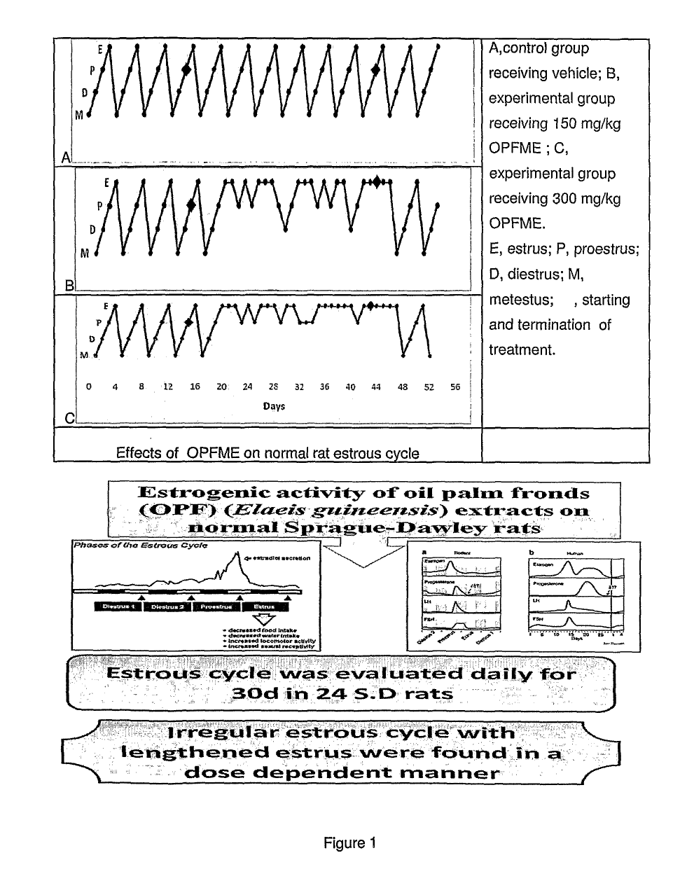

FIG. 2 is a table showing the dose-dependent effect of PALM LEAVES EXTRACT on the uterine weight.

FIG. 3 is a graph showing the effect of PALM LEAVES EXTRACT on vaginal cornification.

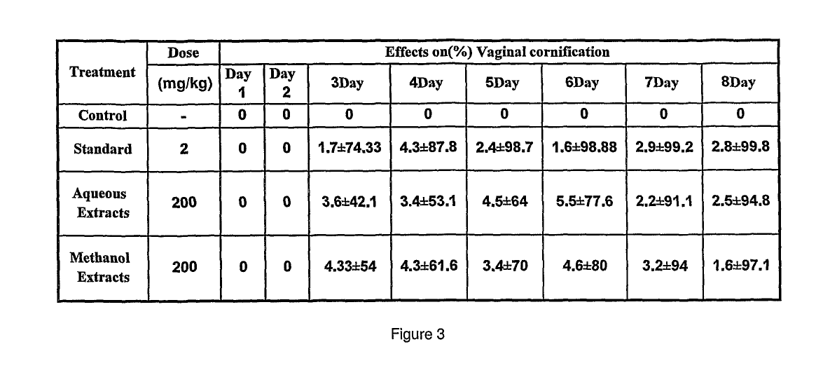

FIG. 4 is a graph showing a comparison of the estrogenic activity of PLE and Premarin, by vaginal cytology assay in ovariectomized rats.

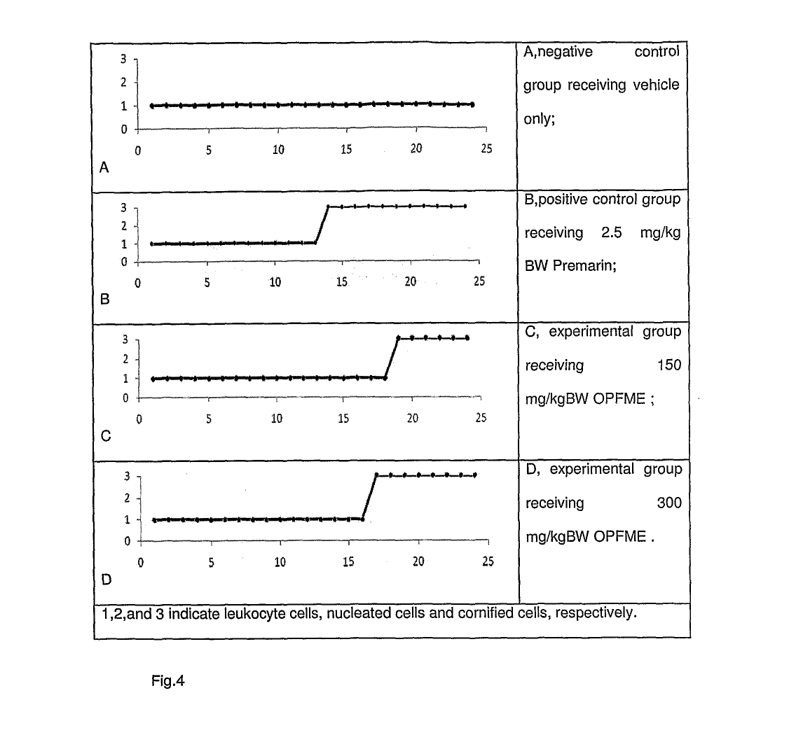

### DETAILED DESCRIPTION OF THE PREFERRED EMBODIMENTS

The present invention relates to a nutraceutical with significant phytoestrogenic property. Hereinafter, this specification will describe the present invention according to the preferred embodiments of the present invention. However, it is to be understood that limiting the description to the preferred embodiments of the invention is merely to facilitate discussion of the present invention and it is envisioned that those skilled in the art may devise various modifications and equivalents without departing from the scope of the appended claims.

The following detailed description of the preferred embodiments will now be described in accordance with the attached drawings, either individually or in combination.

One skilled in the art will readily appreciate that the present invention is well adapted to carry out the objects and obtain the ends and advantages mentioned, as well as those inherent therein. The embodiment describes herein is not intended as limitations on the scope of the invention.

The term “pharmaceutically effective amount” used herein through out the specification refers to the amount of the active ingredient, the extract, to be administered orally to the subject to trigger the desired effect without or causing minimal toxic adverse effect against the subject. One skilled in the art should know that the effective amount can vary from one individual to another due to the external factors such as age, sex, diseased state, races, body weight, formulation of the extract, availability of other active ingredients in the formulation and so on.

It is important to note that the extract used in the disclosed method in this embodiment is derived from the plant species of Arecaceae family. The extracts obtained from the abovementioned plant species are suitable to be incorporated into edible or topical products, or as capsules, ointments, lotions and tablets.

The desired compounds to be extracted from the alcohol/aqueous extracts of are mainly constituted of, but not limited to, biophenols, proteins, lipids, saccharides, minerals and small peptides. Due to polarity of these compounds, the polar solvent such as water, alcohol or acetone is found to be effective in extracting these desired compounds from the plant matrix.

Another embodiment of the present invention involves a comestible and topical composition with phytoestrogenic property comprising alcohol/aqueous extracts derived from the leaves of Palmae family using an appropriate extraction solvent. The comestibles mentioned herein can be any common daily consumed processed food such as bread, noodles, confections, chocolates, beverages, and the like. One skilled in the art shall appreciate the fact that the aforesaid extract can be incorporated into the processed comestibles, capsules, tablets or topical medicine during the course of processing. Therefore, any modification thereon shall not depart from the scope of the present invention.

As setting forth in the above description, the composition with phytoestrogenic properties comprising alcoholic extract from leaves of Palm species. Apart from that the composition may further comprise extract derived from the leaves of Palm species. Preferably, the plant is any one or combination of the plant species of, Elaeis guineensis, Elaeis oleifera, Phoenix dactylifera and Cocos nucifera. The inventors of the present invention found that the alcohol extract derived from the aforementioned species possesses both acceptable taste that confers the derived extract to be comfortably incorporated with the comestibles product, capsules, tablets or topical medicine with minimal additional refining process.

According to the preferred embodiment, the extract to be incorporated into the comestibles and medicine can be acquired from any known method not limited only to the foregoing disclosed method. Following another embodiment, the extract is prepared in a concentrated form, preferably paste or powdery form which enables the extract to be incorporated in various formulations of the comestibles, capsules, tablets or topical products.

In line with the preferred embodiment, the extract shall be the plant metabolites which are susceptible to an extraction solvent. The compounds and small peptides with the phytoestrogenic enhancing and reproductive system health-promoting properties are those metabolites with polarity in the alcohol/aqueous extracts. Therefore, the alcohol/aqueous extracts of leaves of Palmae family is preferably derives from the extraction solvent of water, alcohol, acetone, chloroform, liquid CO₂ and any combination thereof.

In view of the prominent property of promoting phytoestrogenic activity and general healthcare of the reproductive system by the extracts in a subject, further embodiment of the present invention includes a method comprising the step of administrating orally or topically to the subject an effective amount of an extract derived from palm spp. leaves.

The following examples are intended to further illustrate the invention, without any intent for the invention to be limited to the specific embodiments described therein.

### Example 1

Adult female Sprague Dawley rats were randomly divided into three groups containing eight animals in each group: a vehicle control, a low dose (150 mg/kg), and a high dose (300 mg/kg) of palm Leaf extract (PLE) were measured and fed daily in the morning using a 2-g fresh apple wedge as vehicle. The apple vehicle eliminated stress associated with gavage, and rats eagerly ate the apple and the PLE in this manner, making it easy to monitor and ensure complete deliverance of the PLE. Vaginal smears from each rat were monitored daily between 09:00 and 10:00 h using staging criteria described by Everett (Evert. 1989). The vaginal epithelium cells observed under the microscope were classified into three types: leukocyte cells (L), nucleated cells (O) and cornified cells (Co). The representative cell type was determined by choosing the majority of cells. The results of examined vaginal smear cells from five rats in each treatment group were expressed as a mode value (the most frequently occurring cell type in five rats). After 4 week, all rats were removed from treatment.

| Stage | Cell-Types |
|---|---|
| Proestrus | nucleated epithelia |
| Estrus | cornified |
| Metestrus | cornified + leukocytes |
| Diestrus | nucleated + leukocytes |

Based on distribution and density of cell types, each daily vaginal smear was assigned one of four estrous cycle stages: proestrus, estrus, metestrus, and diestrus. Untreated female rats displayed estrus stage for 0.5 days every 4 to 5 d. Both dose of PLE employed produced increased duration of the estrus phase to 2 days (during which ovulation occur) in 75% of the PLE fed animals.

The estrous cycle in females involves many histological, physiological, and morphological and biochemical changes within the ovary. During the estrous cycle the maturation and ovulation of preovulatory follicles takes place under the combined and balanced influence of ovarian and extraovarian hormones. Any imbalance in these hormones leads to irregularity in the function of the ovary and irregular changes in the duration of estrous cycle. Any increase in the duration of estrus phases in the rats indicates further increase of estrogen and FSH levels upon administration of a compound.

### Example 2

Phytoestrogenic enhancing activities of PLE were evaluated in normal and ovariectomised rats at 150 and 300 mg/kg doses. Estrogenic activity of the alcoholic extract was assessed in bilaterally ovarectomized female Sprague-Dawley rats taking percentage vaginal cornification, and uterine wet weight as parameters of assessment. Ovarectomized female rats (9 rats per group) given distilled water were used as a negative control, while rats gavaged daily with 5 mg/kg BW diethylstilbesterol were used as a positive control. At the end of treatment period of 14 days, rats were euthanized; the uteri were dissected and weighed, thereafter.

All rats had only L-type cells throughout the pre-treatment period after ovariectomy which confirmed that the ovaries were completely removed and no endogenous ovarian estrogens were produced. The administration of distilled water did not influence the vaginal epithelium, and only L-type cells were found. In contrast, synthetic estrogen induced a cornification of the vaginal epithelium as early as the third day of treatment. The occurrence of vaginal cornification in rats was dependent the PLE doses with the higher dose showing an earlier response.

Vaginal cytology assay is particularly useful to determine the estrogenic activity of the phytoestrogens, synthetic estrogens or xenoestrogens, based on the specific action of estrogens on specific receptors. Ovariectomised rats, treated orally with estradiol (0.5 mg/day/animal) show vaginal cornification, whereas the sham-treated animals have unestrous vaginal smear. Miroestrol produced cornification of the vaginal epithelium in the immature female mice. Dietary supplementation with phytoestrogens led to increased vaginal cytological maturation in women. Six-month treatment of soy-rich diet to the asymptomatic post-menopausal women increased vaginal cornification of epithelium, karyopycnotic index (KI) and maturation value (MV), identical to those found in the hormonal replacement women.

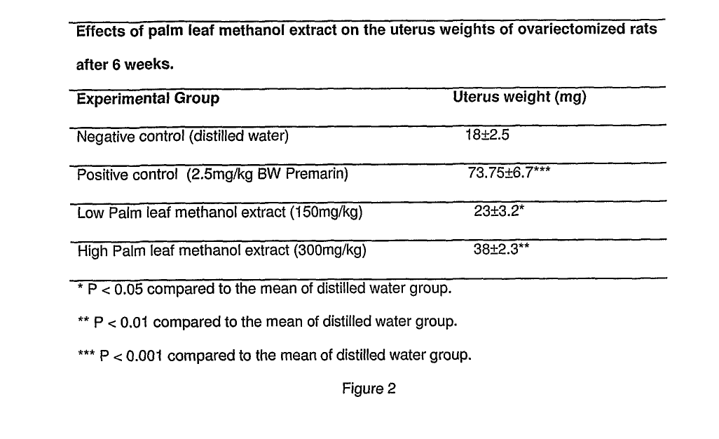

The increment of uterus weight at the end of treatment period in both PLE groups of rats agreed with changes of vaginal epithelium, with the higher doses of PLE producing the heavier uterus weight (FIG. 2).

### Example 3

Forty rats were randomly divided into 5 groups consist of eight rats in each group, control group (normal diet) was considered as negative control. Rats were allowed for acclimatization a week before the induction. Rat mammary gland tumour cells were grown to 80% confluence, harvested using 0.25% Trypsin-EDTA, counted for cell viability using a trypan blue exclusion test, and then resuspended in serum-free media. Cells were inoculated subcutaneously into mammary fat pad (right flank) of female Sprague-Dawley rats with a 200 μl of cell mixture (total 6×10⁷ cells) using a 26-gavage needle. PLE were dissolved in water and administered orally. On day 14, the rats were sacrificed.

All animals were inspected daily, while body weights were recorded weekly. Rats were palpated weekly to monitor tumor development. The diameters of each tumor were measured with calipers and tumor volume was calculated using the following formula:

largest diameter×(smallest diameter)²×0.4 (Li-Qiang et al., 2007).

TABLE 1
Experimental procedures and mammary tumors in Female Sprague Dawley
rats with PLE supplements

| | | | | Post- | | |
|---|---|---|---|---|---|---|
| | | Pre-inoculation | | inoculation | | Total tumour |
| | Acclimation | Treatment | | Treatment | Incidence | Volume |
| Group | (1 week) | (4 week) | Inoculation | (2 week) | (%) | (cm3) |
| Control | + | − | + | − | 87.5% | 10.7 |
| Pre + post | + | + | + | + | 25% | 1.4 |
| inoculation low | | | | | | |
| PLE | | | | | | |
| (150 mg/kg) | | | | | | |
| Pre + Post | + | + | + | + | 12.5% | 0.8 |
| inoculation high | | | | | | |
| PLE | | | | | | |
| (300 mg/kg) | | | | | | |
| Post-Inoculation | + | − | + | + | 37.5% | 2.6 |
| low PLE | | | | | | |
| (150 mg/kg) | | | | | | |
| Post-Inoculation | + | − | + | + | 25% | 0.9 |
| high PLE | | | | | | |
| (300 mg/kg) | | | | | | |

The anticancer activities of the PLE were investigated using a MTT assay on human Breast cancer cell lines (MCF-7). A mitochondrial enzyme in living cells, succinate-dehydrogenase, cleaves the tetrazolium ring, converting the MIT to an insoluble purple formazan.

Cells were exposed to various concentrations of PLE (0-1200 μg/ml) for 48 h. Cells treated with 0.1% DMSO were used as control. The PLE led to proliferation of the MCF-7 cells at 17.5 μg/ml (p<0.05) and an anti-proliferation effect at 150 μg/ml (p<0.05) and 1200 μg/ml (p<0.01) with an IC50 value of 678.5 μg/ml.

### Example 4

PLE enhanced the antioxidant defence system. The hypercholesterol diet triggered a drop in GSH, an increase in lipid peroxidation, and reductions in key antioxidant enzymes activities in the brain, kidney and erythrocytes. In rats, PLE upregulated at least two antioxidant enzymes (SOD and catalase). Although exposure to hypercholesterol diet resulted in oxidative damage of brain, heart, kidney, liver and erythrocyte, PLE counteracted these deleterious effects. PLE administration is associated with an increase antioxidant capacity in blood and organ of rats and also prevented the changes induced by hypercholesterolemia. The reduction in MDA level in hypercholesterolemic rats by PLE showed that PLE prevented lipid peroxidation and this can contribute to a reduction of cardiovascular risk and atherosclerosis.

### Example 5

### Effect of PLE on the Estradiol Levels

Following the 150 mg/kgBW treatment, mean serum estradiol levels were increased from 20.80±4.43 to 33.78±3.2 pg/ml (p=0.09) for 2 wk and to 42.17±6.37 pg/ml (p=0.01) after 4 wk. At a high dose treatment (300 mg/kgBW) mean serum estradiol levels increased from 21.46±2.88 to 35.42±6.37 pg/ml (p=0.05) after 2 wk and to 72.10±8.87 pg/ml (p=0.001) after 4 wk, suggesting a dose dependent effect.

TABLE 2
Antioxidant enzyme level in erythrocyte of Normal and hypercholesterol diet in
Male Sprague Dawley rats with PLE supplements

| | | | | |
|---|---|---|---|---|
| Diets | Normal (N) | N + PLE | High Fat + 1% cholesterol (C) | C + PLE |

Catalase (U/min/mL)

| | | | | |
|---|---|---|---|---|
| Week 0 | 0.012 ± 0.003 | 0.008 ± 0.004 | 0.013 ± 0.016 | 0.005 ± 0.002 |
| 2 | 0.011 ± 0.002 | 0.012 ± 0.003 | 0.013 ± 0.003 | 0.020 ± 0.002 |
| 4 | 0.009 ± 0.001 | 0.008 ± 0.003 | 0.007 ± 0.004 | 0.009 ± 0.002 |
| 6 | 0.010 ± 0.003 | 0.010 ± 0.002 | 0.014 ± 0.001* | 0.011 ± 0.004 |
| 8 | 0.0087 ± 0.003 | 0.010 ± 0.002 | 0.010 ± 0.0005 | 0.010 ± 0.004 |
| 10 | 0.012 ± 0.004 | 0.017 ± 0.004* | 0.010 ± 0.005 | 0.012 ± 0.003 |
| 12 | 0.011 ± 0.003 | 0.005 ± 0.003* | 0.010 ± 0.004 | 0.011 ± 0.003 |

Superoxide Dismutase (U/min/mL)

| | | | | |
|---|---|---|---|---|
| Week 0 | 0.018 ± 0.005 | 0.041 ± 0.038 | 0.025 ± 0.027 | 0.031 ± 0.029 |
| 2 | 0.016 ± 0.002 | 0.029 ± 0.007 | 0.025 ± 0.009 | 0.071 ± 0.011 |
| 4 | 0.013 ± 0.010 | 0.011 ± 0.009 | 0.015 ± 0.003 | 0.014 ± 0.001 |
| 6 | 0.010 ± 0.004 | 0.007 ± 0.002 | 0.004 ± 0.003* | 0.008 ± 0.005 |
| 8 | 0.003 ± 0.003 | 0.004 ± 0.003 | 0.003 ± 0.002 | 0.007 ± 0.005^{#} |
| 10 | 0.003 ± 0.004 | 0.007 ± 0.003 | 0.009 ± 0.002* | 0.007 ± 0.005 |
| 12 | 0.008 ± 0.006 | 0.012 ± 0.006 | 0.013 ± 0.004 | 0.007 ± 0.003^{#} |

Gluthathione Peroxidase (U/min/mL)

| | | | | |
|---|---|---|---|---|
| Week 0 | 13.15 ± 1.60 | 22.10 ± 7.48* | 8.77 ± 4.04 | 5.799 ± 3.45* |
| 2 | 1.02 ± 0.93 | 4.447 ± 1.23* | 11.91 ± 2.87* | 3.69 ± 0.92* |
| 4 | 13.79 ± 3.36 | 6.34 ± 0.48* | 14.43 ± 3.7 | 20.08 ± 3.59* |
| 6 | 39.40 ± 8.59 | 16.37 ± 2.73 | 14.55 ± 4.72 | 7.91 ± 2.43 |
| 8 | 16.23 ± 3.23 | 7.67 ± 1.66 | 18.54 ± 7.85 | 9.56 ± 7.5 |
| 10 | 11.11 ± 3.89 | 9.11 ± 2.23 | 17.13 ± 4.36* | 11.45 ± 3.04^{#} |
| 12 | 3.885 ± 1.25 | 4.48 ± 1.23 | 6.09 ± 1.63* | 3.70 ± 1.27 |

*p < 0.05 versus N control,
^{#}p < 0.05 versus C control

TABLE 3
Malondialdehyde level in organs of Normal and hypercholesterol
diet in Male Sprague Dawley rats with PLE supplements.

| | | | | |
|---|---|---|---|---|
| | | | High Fat + 1% | |
| Diets | Normal (N) | N + PLE | cholesterol (C) | C + PLE |

MDA level (nmol/ml)

| | | | | |
|---|---|---|---|---|
| Brain | 6.44 ± 0.91 | 11.91 ± 2.62* | 15.78 ± 2.94* | 13.09 ± 0.59* |
| Heart | 3.64 ± 1.46 | 4.04 ± 0.92 | 13.98 ± 1.98* | 6.23 ± 0.91^{#} |
| Kidney | 4.73 ± 1.40 | 5.54 ± 0.51 | 7.92 ± 1.21* | 4.20 ± 0.76^{#} |
| Liver | 5.95 ± 0.25 | 5.72 ± 1.29 | 10.6 ± 0.96* | 7.10 ± 0.75*^{#} |
| Lung | 2.94 ± 1.36 | 4.76 ± 0.40 | 3.86 ± 1.12 | 4.65 ± 1.67 |

*p < 0.05 versus N control,
^{#}p < 0.05 versus C control

TABLE 4
Plasma lipid level of Normal and hypercholesterol diet in
Male Sprague Dawley rats with PLE supplements

| | | | | |
|---|---|---|---|---|
| | | | High Fat + 1% | |
| Diets | Normal (N) | N + PLE | cholesterol (C) | C + PLE |

Total cholesterol (mmol/L)

| | | | | |
|---|---|---|---|---|
| Week 0 | 1.38 ± 0.15 | 1.38 ± 0.29 | 1.38 ± 0.11 | 1.35 ± 0.27 |
| 2 | 1.37 ± 0.28 | 1.28 ± 0.18 | 1.67 ± 0.26* | 1.41 ± 0.22 |
| 4 | 1.18 ± 0.09 | 1.13 ± 0.12 | 1.77 ± 0.43* | 1.65 ± 0.39* |
| 6 | 1.37 ± 0.11 | 1.22 ± 0.12 | 2.25 ± 0.48* | 1.89 ± 0.28* |
| 8 | 1.38 ± 0.33 | 1.15 ± 0.11* | 2.55 + 0.37* | 1.49 ± 0.26^{#} |
| 10 | 1.39 ± 0.18 | 0.87 ± 0.11* | 2.52 ± 0.33* | 1.47 ± 0.39^{#} |
| 12 | 1.45 ± 0.2 | 1.12 ± 0.17 | 2.88 ± 0.53* | 1.12 ± 0.36^{#} |

HDL-Cholesterol (mmol/L)

| | | | | |
|---|---|---|---|---|
| Week 0 | 0.57 ± 0.09 | 0.57 ± 0.17 | 0.58 ± 0.07 | 0.55 ± 0.08 |
| 2 | 0.54 ± 0.09 | 0.62 ± 0.09* | 0.70 ± 0.10 | 0.69 ± 0.07 |
| 4 | 0.72 ± 0.20 | 0.75 ± 0.06 | 0.83 ± 0.07 | 0.85 ± 0.05 |
| 6 | 0.53 ± 0.04 | 0.77 ± 0.06* | 0.56 ± 0.10 | 0.96 ± 0.08*^{#} |
| 8 | 0.64 ± 0.1 | 0.77 ± 0.03* | 0.60 ± 0.09 | 0.79 ± 0.14*^{#} |
| 10 | 0.64 ± 0.11 | 0.68 ± 0.02 | 0.65 ± 0.12 | 0.76 ± 0.21^{#} |
| 12 | 0.64 ± 0.19 | 0.70 ± 0.12* | 0.55 ± 0.04* | 0.88 ± 0.19*^{#} |

LDL-Cholesterol (mmol/L)

| | | | | |
|---|---|---|---|---|
| Week 0 | 0.31 ± 0.09 | 0.36 ± 0.17 | 0.27 ± 0.03 | 0.26 ± 0.06 |
| 2 | 0.25 ± 0.09 | 0.27 ± 0.04 | 0.32 ± 0.09 | 0.29 ± 0.08 |
| 4 | 0.24 ± 0.04 | 0.26 ± 0.06 | 0.41 ± 0.06* | 0.41 ± 0.10* |
| 6 | 0.29 ± 0.03 | 0.26 ± 0.02 | 0.52 ± 0.08* | 0.42 ± 0.14 |
| 8 | 0.26 ± 0.006 | 0.26 ± 0.03 | 0.56 ± 0.006* | 0.35 ± 0.08^{#} |
| 10 | 0.25 ± 0.02 | 0.23 ± 0.05 | 0.55 ± 0.02* | 0.29 ± 0.04^{#} |
| 12 | 0.27 ± 0.06 | 0.23 ± 0.03 | 0.57 ± 0.06* | 0.22 ± 0.06^{#} |

Triglycerides (mmol/L)

| | | | | |
|---|---|---|---|---|
| Week 0 | 0.9 ± 0.09 | 0.86 ± 0.08 | 0.90 ± 0.10 | 0.91 ± 0.14 |
| 2 | 0.87 ± 0.11 | 0.81 ± 0.22 | 0.79 ± 0.08 | 0.64 ± 0.10 |
| 4 | 0.75 ± 0.19 | 0.63 ± 0.07 | 0.71 ± 0.18 | 0.60 ± 0.13 |
| 6 | 0.58 ± 0.09 | 0.58 ± 0.10 | 0.71 ± 0.10 | 0.64 ± 0.14 |
| 8 | 0.55 ± 0.08 | 0.55 ± 0.05 | 0.81 ± 0.10* | 0.62 ± 0.03 |
| 10 | 0.43 ± 0.16 | 0.45 ± 0.06 | 0.85 ± 0.09* | 0.47 ± 0.09^{#} |
| 12 | 0.49 ± 0.05 | 0.42 ± 0.05* | 0.86 ± 0.07* | 0.39 ± 0.06*^{#} |

*p < 0.05 versus N control,
^{#}p < 0.05 versus C control

1. A composition with phytoestrogenic property for hormone replacement therapy against oxidative stress, retarding hormone related cancer, affecting fertility and effective to hinder or prevent ailments or conditions resulting from, or exacerbated by, a decrease in endogenous estrogen including: reproductive organ ailments, cardiovascular disease, osteoporosis, loss of cognitive function, urinary incontinence, body fat increase, post menopausal syndromes and vasomotor symptoms, comprising extracts from leaves of palm family.

2. A composition according to claim 1, wherein the Palmae family is preferably derived from any one or combination of the species, Elaeis guineensis, Elaeis oleifera, Phoenix dactylifera and Cocos nucifera.
